(19) United States
(12) Patent Application Publication
(10) Pub. No.: US 2014/0234456 A1
(43) Pub. Date: Aug. 21, 2014

(54) COMPOSITION FOR ANTIDIABETES CONTAINING SMILAX CHINA L LEAVES EXTRACT

(71) Applicant: KANGWON NATIONAL UNIVERSITY University-Industry Cooperation Foundation, Chuncheon (KR)

(72) Inventors: Myeon Choe, Chuncheon (KR); Yun Hwan Kang, Yongin (KR); Dae Jung Kim, Heongseong-Gun (KR); Jae Sung Lee, Chuncheon (KR); Kyong Gon Kim, Sokcho (KR); Sung Mi Lee, Hongseong-Gun (KR)

(73) Assignee: KANGWON NATIONAL UNIVERSITY University-Industry Cooperation Foundation, Chuncheon (KR)

(21) Appl. No.: 14/166,333

(22) Filed: Apr. 9, 2014

(30) Foreign Application Priority Data
Feb. 15, 2013 (KR) 10-2013-0016260

(51) Int. Cl. A61K 36/90

(52) U.S. Cl. CPC A61K 36/90
USPC 424/774

(57) ABSTRACT

An anti-diabetic composition is disclosed. The anti-diabetic composition includes a Smilax china L. leaf extract having no toxicity to human bodies, no side effects such as increase or decrease in body weight, and a very high ability to inhibit the activity of α-glucosidase.

### CROSS-REFERENCE TO RELATED APPLICATION

This application claims priority to Korean Application No. 10-2013-0016260 filed on Feb. 15, 2013, which is incorporated herein by reference.

### TECHNICAL FIELD

The present invention relates to an anti-diabetic composition, and more particularly to an anti-diabetic composition that includes a Smilax china L. leaf extract as an active ingredient and has excellent anti-diabetic effects due to stability in humans, no side effects, and effective suppression of the activity of α-glucosidase.

### RELATED ART

Diabetes mellitus is a chronic disease caused by relatively high blood glucose (sugar) levels due to a lack of insulin, leading to excretion of glucose into the urine. Causes of diabetes mellitus may be deficiency of insulin secreted by B cells of the islets of Langerhans of the pancreas, hyperfunction of endocrine glands such as anterior pituitary, adrenal gland, thyroid, and the like, and lesions of central nerves, in particular hypothalamus, and diabetes mellitus is considered to be caused by combination of these factors.

Diabetes mellitus is classified as type 1 diabetes mellitus (NIDDM) and type 2 diabetes mellitus (IDDM). Type 1 diabetes is insulin-dependent, results from destruction of pancreatic beta cells due to immunological mechanism or insulin production disorder due to abnormal gene expression, and occurs in childhood, while type 2 diabetes is insulin-resistant and insulin non-dependent diabetes in which insulin is normally produced initially but the ability to regulate insulin metabolism is reduced due to insulin resistance and, eventually, insulin secretion disorder of the pancreatic cells occurs, may onset even beyond 40 years of age, is closely related to overweight and obesity, and is referred to as adult onset diabetes.

Currently, the estimated number of patients with diabetes in this country is approximately 2.4 million, which is about 5% of the total population (48 million), and more than half of patients do not recognize that they suffer from diabetes, but are prospective patients that are highly likely to contract diabetes in the near future. Judging from the example of developed countries, the number of patients with diabetes is expected to further increase due to improved income, dietary changes, increasingly sedentary lifestyles, and aging. 90% or more of the patients with diabetes suffer from adult onset type 2 diabetes and an increase in the number of patients with type 2 diabetes tends to make up the majority of increase in the total number of diabetes patients due to aging and changes in lifestyle habits.

Although diabetes occurs for various causes, a basic principle for alleviation or recovery of symptoms regardless of types of diabetes is to reduce time at which glucose is excessively resided in the blood, by increasing the rate at which cells of each of a plurality of tissues use sugar absorbed into the body after meals and is equally applied to drugs or natural substances. To do this, it is necessary to increase enzymatic activity of glucokinase and pyruvate dehydrogenase, involved in intracellular decomposition of sugar and use of a resulting product as a substrate of energy metabolism, to increase enzymatic activity of acetyl-CoA carboxylase involved in conversion of sugar remaining after producing energy into fat, and to suppress enzymatic activity of α-glucosidase. Among these, α-glucosidase is an enzyme involved in carbohydrate digestive process and conversion into glucose and largely contributes to blood sugar increase after meals. Thus, by inhibiting the enzymatic activity of α-glucosidase, digestion and absorption of carbohydrates after meals are delayed and thus blood sugar levels are significantly reduced and, accordingly, insulin concentration in the blood is also decreased, which results in gradually decreasing effects of blood sugar on an empty stomach.

It is very important to develop biomaterials associated with regulation of the enzymatic activity of these enzymes through scientific verification and to develop such biomaterials into functional biofoods, since there are no cure agents for: diabetes. In addition, there is a continuous need to develop a natural treatment with safety, high efficacy, and absent the various side effects of conventional diabetes treatments.

| | Mechanism of | Alc | | |
|---|---|---|---|---|
| | action and | decrease | | |
| | usage | (monotheraphy) | Side effects | Remarks |
| Sulfonylurea | Increase in | 1.0-2.0% | low blood | Hepatopathy |
| (gliclazide, | insulin | | sugar, body | (three times |
| gliplizide, | secretion in | | weight | normal value, |
| glimepiride, | pancreatic | | increase, | renal disease |
| glibenclamide) | beta cells | | arthralgia, | Cr >2.0 mg/dL) |
| | | | arthritis, | |
| | | | lumbago, | |
| | | | bronchitis | |
| Biguanide | Reduction in | 1.0-2.0% | No increase in | <GFR 30 mL/min, |
| (Metformin) | sugar | | body weight | elderly, |
| | production in | | and no low | infection, |
| | liver, | | blood sugar, | dehydration, |
| | improvement in | | digestive | cardiopulmonary |
| | peripheral | | disorders | dysfunction, |
| | insulin | | (loss of | liver failure, |
| | sensitivity, | | appetite, | patients |
| | administration | | nausea, | intravenously |
| | with meals, | | vomiting, | administered |
| | Starting from | | diarrhea), | radiation |
| | small doses | | lactic | iodine |
| | | | acidosis | contract media |
| α-glucosidase | inhibition of | 0.5-0.8% | No increase in | Kidney disease |
| inhibitor | polysaccharide | | body weight | (Cr >2.0 mg/dL), |
| (acarbose, | absorption in | | and no low | liver |
| voglibose, | upper | | blood sugar, | disease, |
| miglitol) | gastrointestinal | | digestive | inflammatory |
| | tract, thus | | disorders | bowel disease |
| | reducing post- | | | |
| | meal high | | | |
| | blood sugar, | | | |
| | administration | | | |
| | prior to meals | | | |
| | (three times | | | |
| | per day) | | | |
| Thiazolidinedione | Improvement in | 0.5-1.4% | Body weight | Severe heart |
| (pioglitazone) | insulin | | increase, | failure or |
| | sensitivity of | | edema, low | patients with |
| | muscles, | | hemoglobin, | personal |
| | liver, and fat | | fracture, | history (NYHA |
| | cells | | heart failure | classes 3 and |
| | | | | 4), hepatic |
| | | | | impairment, |
| | | | | severe renal |
| | | | | impairment |
| Meglitinide | Increase in | 0.5-1.5% | Body weight | Dose |
| (repaglinide, | insulin | | increase, low | adjustment in |
| nateglinide, | secretion, | | blood sugar, | abnormal liver |
| mitiglinide) | reduction in | | constipation, | function |
| | post-meal high | | upper | |
| | blood sugar, | | respiratory | |
| | three times | | tract | |
| | administration | | infection, | |
| | per day before | | sinusitis | |
| | meals | | | |
| DPPIV- | Inhibition of | 0.5-0.8% | No increase in | Kidney |
| inhibitor | Incretin (GLP- | | body weight | disease, no |
| (sitagliptin, | 1, GIP) | | and no low | safety for |
| vildagliptin) | secretion, | | blood sugar | long-term use |
| | glucose- | | | |
| | dependent | | | |
| | insulin | | | |
| | secretion, | | | |
| | inhibition of | | | |
| | glucagon | | | |
| | secretion | | | |
| | after meals | | | |
| GLP-1 | glucose- | 0.5-1.0% | No low blood | No safety for |
| receptor | dependent | | sugar | long-term use |
| agonist | insulin | | weight loss, | |
| (exenatide) | secretion, | | gastrointestinal | |
| | inhibition of | | disorders | |
| | glucagon | | | |
| | secretion | | | |
| | after meals, | | | |
| | inhibition of | | | |
| | gastric | | | |
| | emptying, | | | |
| | subcutaneous | | | |
| | injection | | | |
| | (twice per | | | |
| | day) | | | |

### [Conventional Diabetes Treatment Agents and Side Effects Thereof]

Smilax china L. is a deciduous climbing plant belonging to the order Liliales and the family Liliaceae of monocotyledons and inhabits Korea, Japan, China, etc., fruits and fresh sprouts thereof are edible, and the plant has efficacies such as diuresis, detoxification, stroke, and the like and thus is used for treatment of arthritis, lumbago, furuncle, and the like (Gwansim MOON, Compositions and Usages of Medical Herbs, published by Science Encyclopedia, 1991. etc.). In addition, Smilax china L. contains saponins, alkaloids, phenols, amino acids, organic acids, glucides, essential oils, and the like, seeds and leaves thereof contain crude fat and rutin, and Smilax china L. adsorbs and removes many kinds of heavy metals such as mercury, nickel, cadmium, and the like and, in particular, is known to have an excellent mercury removal efficacy.

Meanwhile, there is disclosure of compositions including a Smilax china L. extract in the related art, e.g., Korean Patent Application Registration No. 10-1086669 (a cosmetic composition for skin whitening including a Smilax china L. extract), Korean Patent Application Registration No. 10-11.56636 (a pharmaceutical composition for preventing or treating blood vessel diseases including a Smilax china L. extract), and Korean Patent Publication No. 10-2012-0100109 (beverage for heavy metal excretion using Smilax china L. and a preparation method thereof). However, there has been no disclosure of a technology regarding the anti-diabetic activity of a Smilax china L. leaf extract.

### SUMMARY

Therefore, the present invention has been made in view of the above problems, and it is an object of the present invention to provide an anti-diabetic composition made of a natural substance and causing no harm to human health and no side effects such as increase or decrease in body weight.

It is another object of the present invention to provide an anti-diabetic composition with very high anti-diabetic effects due to very high inhibitory activity of α-glucosidase when compared to conventional diabetic therapeutic agents.

It is another object of the present invention to provide a food composition for alleviating diabetes including a Smilax china L. leaf extract as an active ingredient.

It is another object of the present invention to provide a pharmaceutical composition for preventing and treating diabetes including a Smilax china L. leaf extract as an active ingredient.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing experimental results of toxicity of a Smilax china L. leaf extract to Raw 264.7 cells;

FIG. 2 is a graph showing experimental results of toxicity of a Smilax china L. leaf extract to mice; and

FIG. 3 is a graph showing measurement results of an ability of a Smilax china L. extract to inhibit the activity of α-glucosidase (SCE_LW: aqueous Smilax china L. leaf extract, SCE_LE: ethanolic Smilax china L. leaf extract, and SCE_R: aqueous Smilax china L. root extract).

### DETAILED DESCRIPTION OF THE INVENTION

The present invention provides a food composition for alleviating diabetes including a Smilax china L. leaf extract or a pharmaceutical composition for preventing and treating diabetes including a Smilax china L. leaf extract.

The food composition for alleviating diabetes or the pharmaceutical composition for preventing and treating diabetes includes a Smilax china L. leaf extract. From results of experimental examples of the present invention, it can be confirmed that the Smilax china L. leaf extract had a higher anti-diabetic efficacy than that of a Smilax china L. root extract. In particular, while the Smilax china L. root extract has a very low inhibitory activity of α-glucosidase, the Smilax china L. leaf extract has a much higher inhibitory activity of α-glucosidase than Acarbose, which is a conventional diabetes therapeutic agent.

In addition, the Smilax china L. leaf extract is not cytotoxic, thus being stable in humans and having no side effects such as increase or decrease in body weight.

In an anti-diabetic composition according to the present invention, the Smilax china L. leaf extract may use ethanol as an extraction solvent. From results of experimental examples of the present invention, it can be confirmed that an ethanolic Smilax china L. leaf extract had a much higher inhibitory activity of α-glucosidase than an aqueous Smilax china L. leaf extract.

In the present invention, Smilax china L. leaves as a raw material for extraction may be used without any treatment. Preferably, Smilax china L. leaves may be used after drying. Drying may be natural drying, hot air drying, freeze-drying, or the like.

In addition, Smilax china L. as a raw material for extraction may be used after pulverization in order to increase extraction efficiency and, more preferably, may be ultramicro-pulverized to 400 to 600 mesh.

In addition, the Smilax china L. leaf extract may be used in a liquid state. In another embodiment, however, the Smilax china L. leaf extract may be used in the form of powder prepared by additional processes such as vacuum distillation, freeze-drying, spray-drying, or the like.

A functional food composition according to the present invention may be formulated in the form of, for example, oral formulation, pouch formulation, or beverages such as drinks, using a method commonly used in the art when used as a health functional food for alleviation of diabetes.

A suitable dose of the food composition may be appropriately set according to formulation, administration method, use, and age, body weight and symptoms of patients administering the food composition. Although not being constant, for example, the amount of active ingredient included in a formulation is generally between 10 and 2000 mg/kg per day for adults. Since the dose of the food composition may vary according to various conditions, an amount smaller than administration amount may be sufficient or an amount exceeding the administration amount may be needed.

The food composition according to the present invention may further include at least one component selected from the group consisting of conventional auxiliaries and additives, and sweeteners such as licorice, vitamin C, citric acid, nicotinic acid, sodium benzoate, aspartame, saccharin, pectin, mannitol, sorbitol, xylitol, guar gum, powdered skim milk, and oligosaccharides and thus preference or taste may be enhanced.

In addition, the pharmaceutical composition according to the present invention may be used as a pharmaceutical composition for prevention and treatment of diabetes. The amount of the Smilax china L. leaf extract included in the pharmaceutical composition for prevention and treatment of diabetes may be adjusted according to usage of preventive and therapeutic agents, conditions of patients to be administered, and types and severity of diseases.

The pharmaceutical composition for prevention and treatment of diabetes may further include a pharmaceutically acceptable carrier, a diluent, or an excipient, in addition to the active ingredient. The pharmaceutically acceptable carrier, the excipient, or the diluent may be at least one selected from among lactose, dextrose, sucrose, sorbitol, mannitol, xylitol, erythritol, maltitol, starch, gum acacia, alginates, gelatin, calcium phosphate, calcium silicate, cellulose, methylcellulose, microcrystalline cellulose, polyvinylpyrrolidone, water, methylhydroxy benzoate, propylhydroxy benzoate, talc, magnesium stearate, and essential oils. In addition, when the preventive and therapeutic agent is a medicine, the preventive and therapeutic agent may further include a filler, an anticoagulant, a lubricant, a wetting agent, a flavor enhancer, an emulsifying agent, a preservative, or the like.

Formulation of the pharmaceutical composition for preventing and treating diabetes may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, extracts, elixirs, ointments, fluidextracts, emulsions, suspensions, decoctions, infusions, ophthalmic solutions, tablets, suppositories, injections, spirits, cataplasma, capsules, creams, troches, tinctures, pastes, pills, and soft or hard gelatin capsules.

A suitable dose of the pharmaceutical composition for preventing and treating diabetes may be determined considering administration methods, age, gender and body weight of users, disease severity, and the like. In an embodiment, the pharmaceutical composition for preventing and treating diabetes may be administered at least once in an amount of 0.1 to 100 mg/kg (body weight) per day based on the amount of the active ingredient. However, the dose of the pharmaceutical composition is provided for illustrative purposes only and may be varied by doctor prescription according to conditions of users.

Hereinafter, the present invention will be described more fully with reference to the following examples, and these examples are provided for illustrative purposes only and are not intended to limit the scope of the invention. In addition, the present invention includes all modifications and equivalents of the technical subject matters of the invention.

### Example

### Preparation of Smilax china L. Leaf Extract

Smilax china L. leaves were washed, freeze-dried, and micro-pulverized. Ethanol was added to the pulverized Smilax china L. leaves in an amount 10 to 20 times the weight of the Smilax china L. leaves, and the resulting mixture was incubated under shaking at 60° C. for 24 hours and then centrifuged at 5,000 to 6,000 rpm for 1 hour. The extract obtained through centrifugal separation was filtered using a 0.45 μm syringe filter and freeze-dried at −80° C. to obtain a powder.

In addition, an aqueous Smilax china L. leaf extract and an aqueous Smilax china L. root extract were prepared using the above-described method and used in the following experimental examples.

### Experimental Example 1

### Experiment for Cytotoxicity of Smilax china L. Leaf Extract

In the present experimental example, cytotoxicity of a Smilax china L. leaf extract was evaluated.

Raw 264.7 cells were cultured in a cell incubator maintained at constant humidity at 37° C. with 5% CO₂ in Dulbecco's modified Eagle's medium (DMEM, Sigma) containing 10% FBS and 2% penicillin/streptomycin. The Raw 264.7 cells were subcultured while the culture medium was replaced with new medium at an interval of three days.

Toxicity of an ethanolic Smilax china L. leaf extract was evaluated using XTT assay kit (2,3-bis(2-methoxy-4-nitro-5-sulfophenyl)-2H-tetrazolium-5-carboxanilide innersalt). The cells were seeded in a 96-well plate at a concentration of 1×10⁴ cells a day before the experiment and the sample was cultured for 24 hours by varying concentration of the sample. Thereafter, XTT and N-methylphenazinium methyl sulfate (PMS) reagents were completely defrosted at 37° C., 1 ml of XTT reagent and 20 μl of PMS reagent were mixed to prepare a working solution, the working solution was collected using a pipet in an amount corresponding to 20% of the volume of the 96-well plate, and the collected working solution was carefully added to each well and mixed while being lightly shaken in a state of the plate being fixed. Thereafter, the resulting 96-well plate was incubated in a CO₂ incubator for 4 hours. Afterwards, cytotoxicity was calculated as a value obtained by subtracting absorbance at 690 nm from absorbance at 450 nm using ELISA (VersaMax ELISA Microplate Reader, Molecular Devices, Sunnyvale, USA).

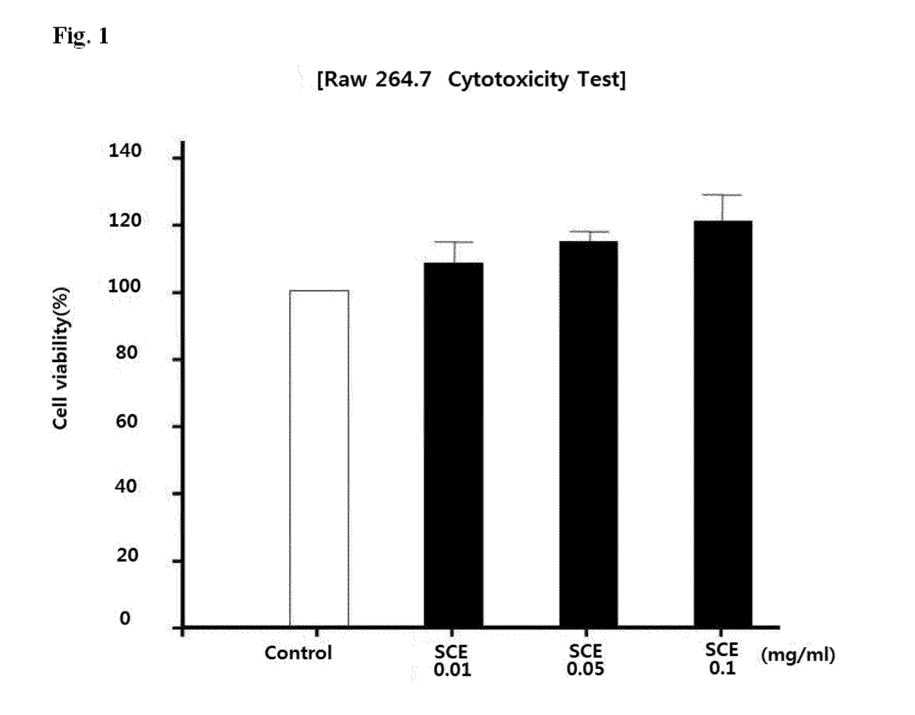

As a result, toxicity of the Smilax china L. leaf extract to the Raw 264.7 cells was not observed (see FIG. 1).

### Experimental Example 2

### Toxicity Experiment of Smilax china L. Leaf Extract on Mice

In the present experiment, toxicity of a Smilax china L. leaf extract on mice was tested.

Five specific pathogen-free (SPF) female balb/c mice and five SPF male balb/c mice as a laboratory animal, obtained from Central Lab Animal, Inc., were separately fed under the following feeding conditions: at 23±2° C., humidity of 50±5%, and 12-hour fluorescent light/12-hour dark cycle. The mice were observed during 1 week acclimation and rearing, only normal animals were used in the experiment after observation, and the experiment was implemented when the laboratory animals were 6 weeks old. Weights of the mice prior to the experiment were measured and recorded, the mice were orally administered an aqueous Smilax china L. leaf extract in a dose of 1.5 g/kg per day while being freely fed a feed and water, and changes in weight prior to or after the experiment were observed. As a control, mice were orally administered a saline solution.

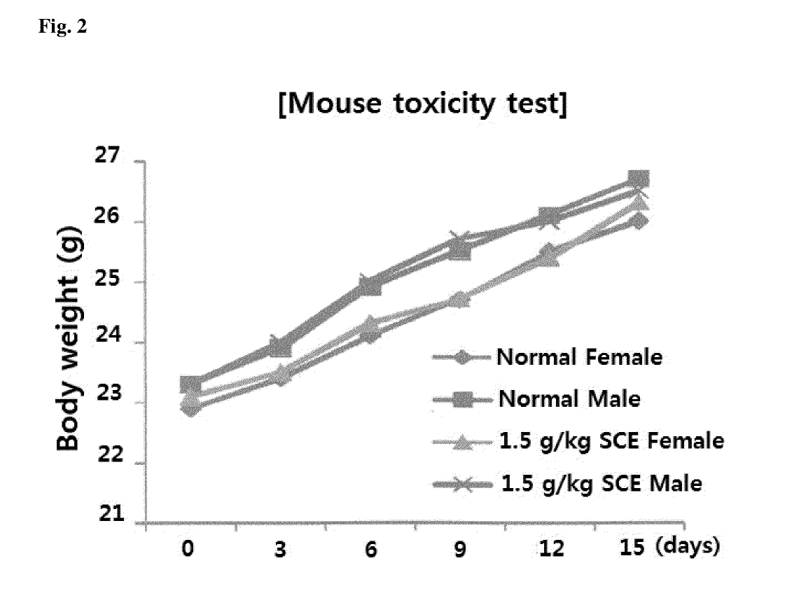

As results of experiment, the Smilax china L. leaf extract had no effect on an increase in weight of the mice. From the experimental results, it can be confirmed that the Smilax china L. leaf extract has no toxicity and no side effects (see FIG. 2).

### Experimental Example 3

### Measurement of Inhibitory Effect of Smilax china L. Leaf Extract on Activity of α-Glucosidase

In the present experimental example, the ability of a Smilax china L. leaf extract to regulate blood sugar was verified by kinetic analysis of an in vitro reaction with a substrate.

2.5 mM p-nitrophenyl α-D-glucopyranoside as a synthetic substrate was added to phosphate buffer (pH 6.8), each of the extract samples (ethanolic Smilax china L. leaf extract, aqueous Smilax china L. leaf extract, and aqueous Smilax china L. root extract) was added thereto, and an enzyme solution was added to the mixed solution. Subsequently, the resulting solution was maintained at 37° C. for 20 minutes to induce reaction therebetween, the reaction was completed by adding 0.1M NaOH thereto, and a degree to which the activity of α-glucosidase is inhibited was evaluated by measuring p-nitrophenol, which is a reaction product released from pNPG as a substrate, at 405 nm using a spectrometer. As a control, acarbose, which is a commercially available diabetes therapeutic agent, was used.

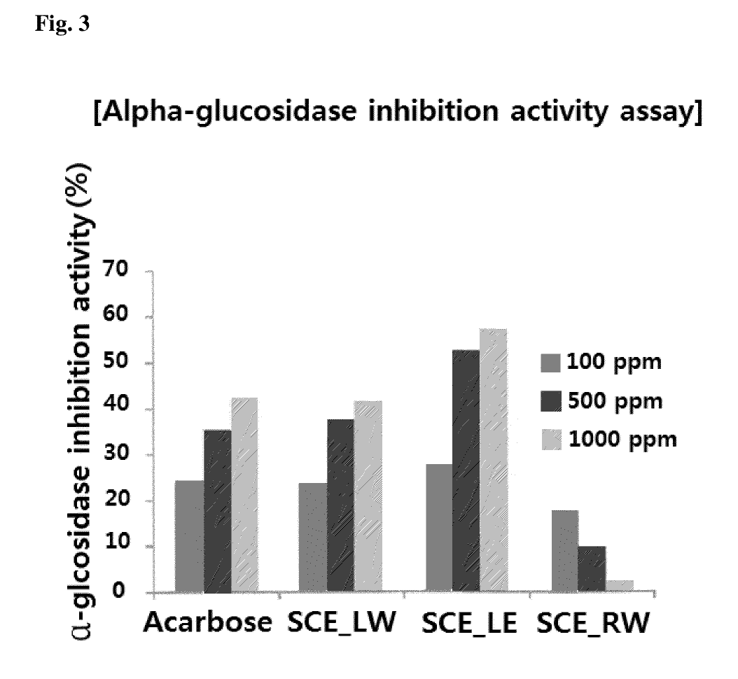

As results of experiment, the Smilax china L. root extract had a very low ability to inhibit the activity of α-glucosidase, while the Smilax china L. leaf extract had a very high ability to inhibit the activity of α-glucosidase. In addition, the aqueous Smilax china L. leaf extract had similar activity to that of acarbose and the ethanolic Smilax china L. leaf extract had much higher activity than that of acarbose (see FIG. 3).

As described above, according to the present invention, an anti-diabetic composition made of a natural substance, having no toxicity in human bodies, and having no side effects such as increase or decrease in body weight may be prepared.

In addition, an anti-diabetic composition with excellent anti-diabetic effects due to much higher α-glucosidase inhibitory activity than that of conventional diabetes therapeutic agents may be prepared.

In addition, according to the present invention, an anti-diabetic functional food composition and an anti-diabetic pharmaceutical composition may be prepared using a Smilax china L. leaf extract with very high anti-diabetic effects.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A food composition for alleviating diabetes comprising a Smilax china L. leaf extract as an active ingredient.

2. The food composition according to claim 1, wherein the Smilax china L. leaf extract uses ethanol as an extraction solvent.

3. A pharmaceutical composition for prevention and treatment of diabetes comprising a Smilax china L. leaf extract as an active ingredient.

4. The pharmaceutical composition according to claim 3, wherein the Smilax china L. leaf extract uses ethanol as an extraction solvent.
